(19) United States
(12) Patent Application Publication
(10) Pub. No.: US 2008/0071079 A1
(43) Pub. Date: Mar. 20, 2008

(54) Process For The Production Of Esters Of Sugars And Sugar Derivatives

(76) Inventors: Kenneth James, Oxon (GB); Frederick James Smith, Oxon (GB)

Correspondence Address: PATENT DOCKET ADMINISTRATOR;LOWENSTEIN SANDLER PC, 65 LIVINGSTON AVENUE, ROSELAND, NJ, 07068, US

(21) Appl. No.: 11/718,058

(22) PCT Filed: Oct. 27, 2005
(86) PCT No.: PCT/GB05/04158

§ 371 Date: Apr. 26, 2007

(30) Foreign Application Priority Data
Oct. 28, 2004 (GB) 0423972.9

(51) Int. Cl. C07H 13/06

(52) U.S. Cl. 536/119

(57) ABSTRACT

A transesterification process for the production of esters of non-reducing sugars or sugar derivatives comprises reacting the sugars or sugar derivatives with a fatty acid alkyl ester in the absence of a solvent and at an elevated temperature, for example from 120 to 135° C., by microwave radiation. The reaction is conducted in the presence of a potassium derivative soluble in the reaction medium, preferably a soap, and especially a soap of a C₁₂ to C₂₂ unsaturated or saturated fatty acid, for example oleic or stearic acid. The reaction proceeds to completion in relatively short time periods, with the result that the process may be conducted in air without the need for a gas blanket or vacuum, and without oxidation of the reactants or reversal of the reaction.

This invention relates to the production of esters of non-reducing sugars or sugar derivatives, and especially, although not exclusively, to sucrose esters.

Esters of sucrose with fatty acids, particularly the sucrose mono-esters and di-esters, are potentially very important materials, and have a number of extremely useful properties. For example, sucrose esters as defined under E473 are non-toxic, odourless, non-irritating to the skin, and when ingested, they hydrolyse to form normal food products. They may, for example, be employed as surfactants, and, unlike most other surfactants are biodegradable under both aerobic and anaerobic conditions. They are very good emulsifiers, and perform well as detergents, either alone or in combination with anionic surfactants, and may be formulated as either high foaming or low foaming detergents. Accordingly, they may be used generally as domestic or industrial detergents, and also in specialized uses such as additives for foodstuffs, for example for treating fresh fruit and vegetables, animal feeds, cosmetics, pharmaceuticals and agricultural chemicals. They may be employed as lubricants, plasticizers (with or without glycerides), emollients, and as emulsifiers. In addition to sucrose esters, sucroglycerides are of considerable commercial importance. Sucroglycerides are commonly mixtures of sucrose esters and glycerides as defined under E474.

However, in spite of possessing such advantages, sucrose esters have never been exploited to their full potential, because of difficulties arising from their production. Many processes have been proposed for their manufacture but because of technical and economic disadvantages, it is still difficult to achieve large-scale industrial production at low cost.

Sucrose esters cannot be prepared by the direct esterification of sucrose with a fatty acid, but may be prepared by transesterification with a fatty acid ester. Most of the known transesterification processes are carried out in a solvent, for example dimethylformamide (DMF) or dimethylsulphoxide (DMSO), and are performed at an elevated temperature in the region of 90° C. in the presence of an alkaline catalyst, for example potassium carbonate, using the methyl ester of the fatty acid.

In the transesterification process, it is necessary to remove water in order to drive the reaction equilibrium in the right direction since the presence of water will cause the reaction to reverse. The water may be removed by heating the system above 100° C. and/or by reduced pressure. In addition, it may be necessary to employ a dry nitrogen blanket in order to prevent traces of water in the air from contaminating the reaction mixture. In the transesterification process it is also preferable to prevent or minimise the ingress of oxygen in order to prevent or minimise oxidation of any unsaturated reactants. The need for anhydrous conditions, the prolonged heating sometimes under reduced pressure, the use of a nitrogen blanket to prevent contamination by water or oxygen and the use of a solvent are serious disadvantages both in terms of the economics of the process, but also because all traces of the solvent must be removed from the product.

Furthermore, the solvent will remain in the reaction product, and such solvent-based processes require the subsequent removal of the solvent if the products are to be employed in foodstuffs. The relatively limited solubility of sucrose in organic solvents also requires a large excess of solvent to be employed, all of which must be removed from the final product and recovered.

It has been proposed to conduct the transesterification reaction without the presence of a solvent, but such processes generally suffer from a number of disadvantages, for example, relatively long reaction times in the order of 8 to 16 hours, relatively low yields, for example in the order of 15 to 20%, or relatively complex and expensive apparatus employing nitrogen or carbon dioxide blankets or conducting the process in a vacuum.

This invention is directed to the use of microwaves in order to conduct the transesterification reaction. A number of patent documents disclose the use of microwaves for this purpose, for example EP-A-0 798 308 (CECA S.A.) which describes reacting dianhydro-1,4:3,6-D-glucitol with methyl dodecanoate in a dimethylformamide solvent under the action of microwaves.

WO 03/090669 (Aldivia S.A.) describes a method for the production of esterified polyhydroxylated alcohols, for example sorbitol, mannitol or xylitol, by esterification, transesterification or interesterification using microwaves in an atmosphere deprived of oxygen.

GB-A-2,361,918 (Interpole Ltd.) describes a process for the transesterification of sucrose using a NaOH catalyst under vacuum and employing microwaves, which purports to generate the octaester.

According to one aspect, the present invention provides a process for the production of an ester of a non-reducing sugar or sugar derivative, which comprises reacting the sugar or sugar derivative with a fatty acid alkyl ester at an elevated temperature, wherein the reaction is effected by means of microwave radiation and is conducted in the presence of a potassium soap.

The term “non-reducing sugar derivative” is intended to mean that sugar derivative, rather than the sugar from which it is formed, is not oxidized by reagents such as Fehling's solution etc. Thus, the sugar derivative may be formed from a reducing sugar provided that any aldehyde or keto group in the sugar has been protected or removed in forming the derivative.

The process may be employed to produce esters of any of a number of non-reducing sugars or sugar derivatives. Advantageously the non-reducing sugar or sugar derivative comprises a non-reducing disaccharide, a glycoside of a mono- or disaccharide, or a polyol that has been formed by reduction of a mono- or disadcharide. Thus, sucrose or trehalose may be used, especially sucrose. Preferred sugars for forming the glycosides include ketoses such as fructose, sorbose, tagetose, psicose; pentoses such as lxyose, ribose, arabinose or xylose; aldoses such as allose, altrose, glucose, mannose, gulose, idose, galactose or talose; or C₄ sugars such as erythrose or threose. The glycosides may be formed from straight-chain or branched lower (C₁ to C₆) alkanols, preferably methanol, ethanol or propanol.

Any of the reducing sugars may be employed to form a polyol, sorbitol, mannitol and lactitol being preferred.

We have determined, as described in more detail below, that although GB-A-2,361,918 purports to generate sucrose octaester by transesterification with methyl palmitate, no such ester is formed under the reaction conditions described therein or even when longer times, higher temperatures or more catalyst is used. What is important to the formation of esters is the fact that the reaction is conducted in the presence of a potassium soap.

The reason why the presence of the potassium soap is important to the reaction is not understood. The soap is a source of readily available potassium ions as well as acting as an emulsifier, which will increase the solubility of the sugar or sugar derivative in the ester of the fatty acid, but the ability of the soap to act as an emulsifier does not explain the dramatic effect of the presence of the soap to the reaction or why this effect is specific to potassium. The soap will typically be formed from a fatty acid having a straight-chain or branched, saturated, mono-unsaturated or poly-unsaturated alkyl group having at least 6, preferably at least 12 carbon atoms, but normally not more than 22 and especially not more than 18 carbon atoms.

Preferably, although not necessarily, the reaction will be conducted substantially in the absence of a solvent and in air. It is possible to include some solvent in the reaction mix, although there will be no advantage to this and the presence of a solvent will have the disadvantage that the solvent will need to be removed. Similarly, it is possible to employ an inert gas blanket or a vacuum, but this also is not necessary and some of the advantage of the invention will thus be lost in terms of a simplified process.

By the phrase “in air” is meant that the process is conducted in the atmosphere without any inert gas being provided or without the reaction being conducted under a vacuum, in order to prevent atmospheric moisture or oxygen reaching the reactants. It is not necessary for the reaction to be conducted at atmospheric pressure: super- or sub-atmospheric pressures may be employed if desired, but no special techniques or precautions are required. Typically the process will be conducted at pressures above 500 mbar.

The process according to the invention has the advantage that it is possible to conduct the reaction to produce a relatively high yield in a relatively short period of time, for example in less than 5 hours, typically from 1 to 5 hours. The reduction in length of time for the reaction enables the reaction to be conducted in the presence of air without atmospheric oxygen causing excessive degradation of the unsaturated components of the reaction mix and so the reaction may be performed without the need to provide a vacuum or an inert gas blanket.

The process according to the invention is conducted at an elevated temperature, but this should not be so high as to initiate degradation of the reactants and consequential colour formation. Thus, as will be appreciated, the process will normally employ heterogeneous reaction conditions in which the sucrose and the alkyl ester reactants are present as separate phases.

The use of microwave radiation has the significant advantage that the temperature of the reaction mixture may be controlled very precisely, for example by employing closed loop feedback control. The process is preferably conducted within a relatively narrow temperature band, for example from 120 to 140° C., and preferably from 125 to 135° C., in the case of the preparation of sucrose esters. If the temperature is significantly below 120° C., the reaction will not proceed sufficiently quickly to enable a worthwhile yield to be obtained, while if the temperature is allowed to rise significantly above 140° C., there is a danger that the reactants will degrade, causing a discoloured reaction product.

We have found that the reactant mixture is capable of undergoing the transesterification reaction at the normal frequency range provided by a domestic microwave oven, typically 2.45 GHz. Absorption of the microwave energy takes place even though the reactants are of low dielectric constant and loss factor and are usually anhydrous. For example, the dielectric constants of the reactants are:

Methyl palmitate: ε=3.1 at 40° C.

Sucrose: ε=4.3 at 25° C.

whereas the typical reaction solvent (water) and component of food that absorbs microwave energy, has a dielectric constant ε of 80 at 20° C. The reaction temperature can be attained smoothly and can be controlled easily, and, as a consequence, the reaction proceeds smoothly and rapidly.

Without the use of microwave radiation, maintenance of an elevated temperature will require heat flow into the reaction mix, which will result in temperature gradients to be formed and may therefore cause hot spots which can cause degradation of the reactants and products even if the recorded temperature falls within the allowed temperature range. Preferably, the reaction is conducted while stirring the reactants, and especially while stirring them, preferably continuously, in order to minimise the temperature differences within the reaction medium. It is thus possible to obtain a product that is light in colour and does not require decolourisation or bleaching.

The fatty acid alkyl ester may have a straight-chain or branched fatty acid alkyl group which may be mono- or poly unsaturated, and preferably have a length of at least 6, and especially at least 12 carbon atoms, but usually no more than 22 and preferably no more than 18 carbon atoms. The alkyl ester may be formed from a fatty acid and a monohydric alcohol or a polyol, preferably an alcohol having a lower alkyl group, for example having up to six carbon atoms, and especially methanol, ethanol or glycerol.

Normally, the reaction will be conducted in the presence of one or more alkaline catalysts. The catalyst may be any of the basic compounds conventionally used as transesterification catalysts, but potassium carbonate and sodium methoxide are preferred. Other basic compounds such as ternary or quaternary organic bases, silicates and borates may also be used. The catalyst will normally be present in a quantity of up to 12%, especially from 3 to 12% by weight of the reaction mix, although quantities outside this range may be employed.

Normally the reaction mix will contain at least 0.1 mole of the non-reducing sugar or sugar derivative, per mole of alkyl ester, but usually not more than 2 moles of sugar or sugar derivative per mole of alkyl ester.

The microwave radiation may have any of a number of frequencies, although it has been found that radiation of 2.45 GHz frequency normally employed in domestic microwave apparatus is effective for promoting the reaction. The radiation may be pulsed or continuous, and will preferably be employed in a range of from 120 to 2000 W per kg of reaction mix.

The crude reaction product will normally contain a mixture of esters of the non-reducing sugar or sugar derivatives, unreacted sugar or derivative, unreacted alkyl fatty acid esters, catalyst and soaps. The esters of the sugar or sugar derivative will need to be extracted from the reaction mixture. A solvent extraction method is preferably employed in which different solvents in which the various reaction products are soluble are used. For example, a solvent in which sucrose is insoluble, such as a lower (e.g. C₁-C₆) alkanol, may be used to separate the sucrose esters and alkyl esters from unreacted sucrose, followed by a further solvent extraction step using a solvent in which either the alkyl ester or the sucrose ester component is soluble in order to separate the two.

In one preferred process, the reaction mix is treated with sec-butanol to separate sucrose from the other materials. The extraction may be employed at room temperature while stirring, and employing from 2 to 10 parts of solvent, preferably from 3 to 5 parts of solvent, and especially about four parts of solvent per part of reaction mix. Insoluble material, mainly sucrose, may be removed by filtration or, more preferably, by centrifugation, and may be reused. If desired, an ion exchange resin may be employed, to convert any soaps to free fatty acids in which case it is convenient to add the ion exchange resin at this stage. This enables the free fatty acids to be extracted with the unreacted methyl esters.

The liquid phase will contain, apart from the solvent, the sucrose esters and the alkyl ester reactant employed for the transesterification. After removal of the solvent, for example by evaporation, the sucrose esters and the alkyl ester may be separated by a further solvent extraction step, for example using a solvent such as ethyl acetate in which the alkyl ester and free fatty acids if present are soluble. Typically from 2 to 10 parts of solvent, preferably from 3 to 5 parts of solvent, and especially about 4 parts of solvent will be employed per part of the solid phase. In addition, it is preferred for the solvent to be cold, for example at a temperature of not more than 5° C., and preferably at about −5° C. In this case, the solid phase will contain substantially only the sucrose esters which may be employed if desired without further processing other than drying if necessary.

The solvent may be removed from the liquid phase for example by evaporation, and both the solvent and the alkyl ester may be recycled.

The following Examples illustrate the invention:

Methyl palmitate and cocoate were prepared from commercially available palm or coconut oil by reaction with methyl alcohol using either p-toluene sulphonic acid or sodium methoxide as the catalyst.

Creation of Crude Sucrose Ester Reaction Product

### EXAMPLE 1

158 grams (approximately 0.55 moles) of Methyl palmitate from naturally occurring palm oil (with an assumed formula CH₃(CH₂)₁₄COOCH₃) was mixed with potassium oleate (7.4 grams 60% solids) and heated to 110° C. using microwave radiation to drive off excess water. The resulting mixture was then added to a dry powder blend of comminuted sugar 90 grams (approximately 0.26 moles), potassium carbonate (5.0 grams) and sodium methoxide (7.4 grams) and mixed thoroughly using a high shear mixer. The reaction mass was then transferred to a domestic microwave oven fitted with a top entry low shear mixer and a microwave source operating from the side of the oven, and pulsed at approximately 2 minute intervals either on low or defrost setting until the temperature reached 125° C. Pulsed radiation on a low setting was continued for 4 hours maintaining the temperature between 125 and 135° C., while stirring the reaction mix continuously. Samples were taken and were analysed by T.L.C. analysis visualizing the reaction products with concentrated sulphuric acid in ethyl alcohol and heating at 110° C. Ester formation was observed after 1, 2 and 3 hours with significant ester formation after 3 hours. The conditions were maintained for a further 1 hour, after which time the reaction was stopped yielding a soft light brown waxy material.

### EXAMPLE 2

Example 1 was repeated using 190 grams of methyl palmitate (approximately 0.66 moles) and 90 grams (0.25 moles) of sucrose to yield a soft light brown waxy material after 4 hours.

### EXAMPLE 3

Example 1 was repeated using 130 grams of methyl cocoate (approximately 0.63 moles) and 80 grams (approximately 0.23 moles) of sucrose to yield a light stiff waxy material after 4 hours.

### EXAMPLE 4

90 grams of sucrose (approximately 0.25 moles) was reacted with 160 grams of refined, deodorised palm oil (approximately 0.18 moles) in the presence of potassium carbonate catalyst, sodium methoxide and potassium oleate using pulsed microwave radiation from a domestic microwave oven with stirring, while maintaining the temperature between 125-135° C. The reaction was complete after 4 hours.

Extraction of Crude Reaction Product to Determine Sucrose Ester Content

### EXAMPLE 5

The reaction product from Example 1 (40 grams) was stirred with sec-butyl alcohol (160 grams) at room temperature for 10 minutes, and the resulting slurry filtered. The residue was dried to yield 12.06 grams of a sticky powder consisting of sucrose and some soaps.

The filtrate was evaporated to dryness to yield a mixture of sucrose esters, methyl esters and soaps as a viscous oil (27.68 grams). The oil was extracted with cold ethyl acetate (114 grams at −5° C.) and filtered. The filtrate was evaporated to dryness to yield a mobile, light coloured oil (9.64 grams) consisting of methyl esters. The residue was dried to yield sucrose esters and some soaps, yield 15.71 grams, 39% on reaction mass.

### EXAMPLE 6

The reaction products from Example 3 (40 grams) was extracted with sec-butyl alcohol (160 grams) at room temperature and filtered. The filtrate evaporated to dryness to yield a viscous oil containing sucrose esters, methyl esters and soaps (weight 31.96 grams). The weight of butyl alcohol insoluble product (sucrose) was 10.82 grams.

The sec-butyl alcohol soluble material was extracted with cold ethyl acetate at −5° C. and filtered. The weight of ethyl acetate soluble material (methyl esters) was 11.04 grams, and the weight of ethyl acetate insoluble product (sucrose esters plus soaps) was 20.92 grams (52.0% on reaction mass).

### EXAMPLE 7

Example 6 was repeated with the exception that an ion exchange resin (Amberlite IRC (trademark) sold by Rohm & Haas) in the acid form was added to the sec-butyl alcohol extraction to convert any soaps to free fatty acids and hence make them soluble in ethyl acetate. The extraction procedure was continued as described in Example 5 to yield 18.49 grams of sucrose esters (46% reaction mass).

### EXAMPLE 8

The reaction mass from Example 3 (methyl cocoate) (40 grams) was extracted with cold ethyl acetate (160 grams) and filtered. The weight of ethyl acetate soluble material (methyl esters) was 13.4 grams. The ethyl acetate insoluble material (25.94 grams) was extracted with sec-butyl alcohol (100 grams) and filtered to yield sucrose cocoate esters. The weight of the residue (sucrose plus some soaps) was 11.75 grams, and the weight of sec-butyl alcohol soluble material (sucrose esters) was 14.4 grams (34.2% on reaction mass).

### EXAMPLE 9

The reaction mass from Example 1 (40 grams) was extracted with sec-butyl alcohol (160 grams) and filtered. The residue, comprising sucrose and some soaps, was dried to yield a solid mass weight of 11.39 grams.

The filtrate was treated with an ion exchange resin (Amberlite IRC-50 (trademark) from Rohm & Haas), (H+form) and evaporated to dryness. The weight was 28.75 grams.

The residue of sucrose, methyl esters and fatty acids was extracted with 130 grams of cold ethyl acetate (−5° C.) and filtered. Residue of sucrose esters was dried to yield 18.20 grams (46% on reaction mass). The filtrate was then evaporated to dryness to yield free fatty acids and methyl esters (weight 10.45 grams).

### EXAMPLE 10

The reaction mass from Example 1 (40 grams) was extracted with 160 grams of cold ethyl acetate (5° C.) and filtered. The residue of sucrose esters, sucrose and soaps was dried to yield 29.19 grams. Filtrate was evaporated to dryness to yield 11.81 grams of methyl esters. The residue was then extracted with sec-butyl alcohol 120 grams and filtered. The residue of sucrose and some soaps was dried to give 11.81 grams by weight.

The filtrate was evaporated to dryness to yield 17.74 grams of the sucrose esters (44% on reaction mass).

### EXAMPLE 11

The reaction mass from Example 4 (40 grams) was allowed to cool and suspended in warm dry isopropyl alcohol (200 grams). The resulting suspension was filtered to remove unreacted sucrose, and the filtrate treated with anhydrous calcium chloride. The precipitate of calcium soaps, potassium and sodium chloride was removed by filtration and the filtrate evaporated to dryness to yield a soft waxy mass consisting of sucrose esters, mono-, di- and triglycerides (30 grams corresponding to 75% on reaction mass (sucroglycerides)).

### EXAMPLE 12

The cooled reaction mass from Example 4 (40 grams) was extracted with cold ethyl acetate (160 grams) and filtered. The residue of sucrose, sucrose esters and soaps was dried and extracted with warm dry isopropyl alcohol (160 grams) containing anhydrous calcium chloride. The precipitate (calcium soaps, potassium and sodium chloride) was removed by filtration and the filtrate evaporated to dryness, yielding a soft waxy mass of sucrose esters of 18 grams corresponding to 45% of the reaction mass.

### REPEAT OF EXAMPLE N.8 OF GB-A-2,361,918

### EXAMPLE 13.1

Example N.8 of GB-A-2,361,918 was repeated (342 grams sucrose, 2,160 grams methyl palmitate and 0.5 grams NaOH) using a domestic microwave and with the exception that the temperature was increased to 120° C. rather than 100° C. specified in Example N.8 (it is well known in the art that no reaction would be expected at 100° C. in the absence of a solvent). Samples were taken every hour for the specified time and for a further two hours, and analysed by T.L.C. in order to determine whether any sucrose ester could be detected. No sucrose ester formation could be detected.

### EXAMPLE 13.2

Example 13.1 was repeated with the exception that the quantity of NaOH catalyst was increased 20 fold (10 grams) and samples were taken and analysed every hour for the specified time and for a further two hours. No sucrose ester formation could be detected.

### EXAMPLE 13.3

Example 13.2 (increased quantity of catalyst) was repeated with the exception that the temperature was increased from 120° to 125-130° C. and samples were taken and analysed every hour for the specified time and for a further two hours. No sucrose ester formation could be detected.

### EXAMPLE 13.4

Example 13.1 was repeated with the exception that methyl palmitate was replaced with methyl stearate. Samples were taken and analysed by T.L.C for the specified time and for a further two hours. No sucrose ester formation could be detected.

Determination of Reactant Necessary for Ester Formation.

### EXAMPLE 14

Example 1 was repeated with the exception that no potassium oleate was present. The reaction was continued for 3 hours at 125-130° C. and samples were taken and analysed by T.L.C. No sucrose ester formation was observed.

### EXAMPLE 15

Example 14 was repeated with the exception that pure (96%) methyl palmitate in place of natural methyl palmitate. The reaction was continued for 4 hours at 125-130° C. and samples were taken and analysed by T.L.C. No sucrose ester formation was observed.

### EXAMPLE 16

Example 1 using pure (96%) methyl palmitate was repeated with the exception that the potassium oleate was replaced with methyl oleate. The reaction was continued for 4 hours at 125-130° C. and samples were taken and analysed by T.L.C. No sucrose ester formation was observed.

### EXAMPLE 17

Example 1 was repeated with the exception that the methyl palmitate was replaced with technical (60%) methyl oleate and that no potassium oleate was present. The reaction was continued for 4 hours at 125-130° C. and samples were taken and analysed by T.L.C. No sucrose ester formation was observed.

In each of Examples 14 to 17, the sucrose tended to form a hard mass. It was concluded that a metal soap was necessary for sucrose ester formation rather than sucrose ester formation being caused by the fatty acid anion or by any other components in the natural esters.

Determination of Scope of the Metal Soap

### EXAMPLES 18 TO 32

Example 1 was repeated employing a range of metal soaps in the reaction mixture. 0.26 moles of sucrose and 0.55 moles of methyl esters were employed in each case. 0.13 moles of soaps of group I metals, 0.065 moles of soaps of group II metals, and 0.044 moles of soaps of group III metals were employed in order to give the same concentration of soap anion, and the temperature was maintained at a range of 120 to 140° C. Ester formation was determined by T.L.C. as described in Example 1. The results are shown in Table 1

TABLE 1

| | | | Sucrose ester |
|---|---|---|---|
| Example | Soap | Ester | formation |
| 18 | K acetate | Me oleate | No |
| 19 | K acetate + Na | Me oleate | No |
| | oleate | | |
| 20 | K citrate + Na | Me oleate | No |
| | oleate | | |
| 21 | Na oleate | Me oleate | No |
| 22 | Na oleate | Me palmitate | No |
| 23 | Na oleate | Palm oil¹ | No |
| 24 | Na oleate | HPKO² | No |
| 25 | K stearate | Me oleate | Yes |
| 26 | K stearate | Me stearate | Yes |
| 27 | Na stearate | Me oleate | No |
| 28 | Ca stearate | Me oleate | No |
| 29 | Ca oleate | Me oleate | No |
| 30 | Li stearate | Me oleate | No |
| 31 | Li oleate | Me oleate | No |
| 32 | Al distearate | Me oleate | No |

¹triglyceride of C₁₆-C₁₈ fatty acids
²Hydrogenated palm kernel oil (c. C₁₂)

It can be seen from the table that soaps of metals other than potassium do not lead to sucrose ester formation, nor do potassium salts of short-chain carboxylic acids such as acetic acid or citric acid, even when sodium oleate is added as an emulsifying agent. Processes in which potassium oleate is employed appear to lead to sucrose ester formation more rapidly than when potassium stearate is used, and it is conjectured that this may be because of the increased solubility of potassium oleate in the reaction medium.

Formation of Esters of Sugar Derivatives

### EXAMPLES 33 TO 36

Example 1 was repeated employing lactitol, sorbitol and methyl glucoside in place of sucrose, and at the same molar quantity as the sucrose in Example 1 (0.26 moles). The methyl ester employed was methyl oleate or methyl stearate depending on the potassium soap used. The temperature was maintained at a range of 120 to 140° C., and ester formation was determined by T.L.C. as described in Example 1. The results are shown in Table 2, from which it can be seen that ester formation of the sugar derivative was observed in all cases.

TABLE 2

| | | Sugar | Methyl | Ester |
|---|---|---|---|---|
| Example | Soap | Derivative | Ester | formed |
| 33 | K oleate | sorbitol | Me oleate | yes |
| 34 | K oleate | lactitol | Me oleate | yes |
| 35 | K oleate | Me glucoside | Me oleate | yes |
| 36 | K stearate | lactitol | Me stearate | yes |

1. A process for the production of an ester of a non-reducing sugar or sugar derivative, which comprises reacting the non-reducing sugar or sugar derivative with a fatty acid alkyl ester substantially in the absence of a solvent and in air at an elevated temperature, wherein the reaction is effected by means of microwave radiation and is conducted in the presence of a potassium soap.

2. A process as claimed in claim 1, wherein the sugar or sugar derivative comprises a non-reducing disaccharide, a glycoside of a mono- or disaccharide, or a polyol formed by reduction of a mono- or disaccharide.

3. A process as claimed in claim 2, wherein the non-reducing disaccharide is sucrose or trehalose.

4. A process as claimed in claim 2 or claim 3, wherein the glycoside is a glycoside of fructose, sorbose, tagetose, psicose, xylose, ribose, arabinose, lyxose, allose, altrose, glucose, mannose, gulose, idose, galactose, talose, lactose or maltose.

5. A process as claimed in claim 4, wherein the glycoside is a glycoside of fructose or glucose.

6. A process as claimed in claim 4 or claim 5, wherein the glycoside has been formed by reacting the sugar with an alcohol having a straight-chain or branched alkyl group of from 1 to 6 carbon atoms.

7. A process as claimed in any one of claims 2 to 6, wherein the polyol is formed by reduction of fructose, sorbose, tagetose, psicose, xylose, ribose, arabinose, lyxose, allose, altrose, glucose, mannose, gulose, idose, galactose, talose, lactose or maltose.

8. A process as claimed in any one of claims 1 to 7, wherein the fatty acid alkyl ester has a straight-chain or branched, saturated, mono- or polyunsaturated fatty acid chain having a length in the range of from 6 to 22 carbon atoms.

9. A process as claimed in claim 8, wherein the fatty acid alkyl ester has a fatty acid chain in the range of from 12 to 18 carbon atoms.

10. A process as claimed in any preceding claim, wherein the fatty acid alkyl ester is an ester of a fatty acid with methyl, ethyl or propyl alcohol, or glycerol.

11. A process as claimed in any preceding claim, which is conducted in the presence of an alkaline catalyst.

12. A process as claimed in claim 5, wherein the alkaline catalyst is present in the range of from 3 to 12% of the reaction mix.

13. A process as claimed in claim 11 or claim 12, which is conducted in the presence of a mixture of alkaline catalysts.

14. A process as claimed in any one of the preceding claims, wherein the potassium soap has a chain length in the range of from 6 to 22 carbon atoms.

15. A process as claimed in claim 14, wherein the potassium soap has a of chain length of at least 12 carbon atoms.

16. A process as claimed in claim 14 or claim 15, wherein the potassium soap has a chain length of not more than 18 carbon atoms.

17. A process as claimed in any one of the preceding claims, wherein the potassium soap has an unsaturated fatty acid chain.

18. A process as claimed in any one of the preceding claims, wherein from 0.1 to 2 moles of sucrose are employed per mole of fatty acid alkyl ester.

19. A process as claimed in any one of the preceding claims, which is conducted at a temperature in the range of from 120-135° C.

20. A process as claimed in any preceding claim which is conducted for a period of up to 5 hours.

21. A process as claimed in any one of the preceding claims, wherein the reaction mix is continuously stirred in order to maintain a relatively even temperature.

22. A process as claimed in any one of the preceding claims, which includes the step of isolating the ester of the non-reducing sugar or sugar derivative from the reaction mix by means of solvent extraction.

23. A process as claimed in claim 22, wherein the solvent comprises an alkyl ester, a ketone, or an alcohol.

24. A process as claimed in claim 22, wherein the solvent comprises ethyl acetate, isopropanol, sec-butanol, or methyl ethyl ketone.

25. A process as claimed in any one of the preceding claims, wherein the microwave radiation is applied at a power in the range of from 120 to 2000 W per kg of reaction mix.
